(19) United States
(12) Patent Application Publication
(10) Pub. No.: US 2025/0092387 A1
(43) Pub. Date: Mar. 20, 2025

(54) METHOD AND APPARATUS FOR OPTIMIZING MRNA SEQUENCE, MRNA MOLECULE, PHARMACEUTICAL COMPOSITION AND USES THEREOF

(71) Applicant: BEIJING BAIDU NETCOM SCIENCE TECHNOLOGY CO., LTD., Beijing (CN)

(72) Inventors: Yang LIU, Beijing (CN); Xiaomin FANG, Beijing (CN); Jie GAO, Beijing (CN); Xiaonan ZHANG, Beijing (CN); Jingzhou HE, Beijing (CN)

(73) Assignee: BEIJING BAIDU NETCOM SCIENCE TECHNOLOGY CO., LTD., Beijing (CN)

(21) Appl. No.: 18/968,907

(22) Filed: Dec. 4, 2024

(30) Foreign Application Priority Data
Sep. 30, 2024 (CN) 202411390778.8

(51) Int. Cl. C12N 15/10

(52) U.S. Cl. CPC C12N 15/1089

(57) ABSTRACT

A method and an apparatus for optimizing an mRNA sequence, an mRNA molecule, a pharmaceutical composition, and a use thereof are provided. The disclosure relates to the technical field of artificial intelligence, specifically to technical fields such as biological computing. The method for optimizing the mRNA sequence include: obtaining a first mRNA sequence for synthesizing a protein of interest, where the first mRNA sequence includes a 5′ untranslated region sequence and a coding region sequence; and adjusting the 5′ untranslated region sequence and the coding region sequence with the goal of maximizing a first score of the first mRNA sequence, so as to obtain an optimized second mRNA sequence for synthesizing the protein of interest, where the first score reflects at least one of the following indicators of the first mRNA sequence: translation initiation efficiency, codon adaptation index, and minimum free energy.

### CROSS REFERENCE TO RELATED APPLICATION

This application claims priority to Chinese Patent Application No. 202411390778.8, filed on Sep. 30, 2024, the contents of which are hereby incorporated by reference in their entirety for all purposes.

### TECHNICAL FIELD

The present disclosure relates to the technical field of artificial intelligence, especially to technical fields such as biological computing, and specifically relates to a method and apparatus for optimizing an mRNA sequence, an electronic device, a computer-readable storage medium, a computer program product, an mRNA molecule, a pharmaceutical composition and uses thereof.

### BACKGROUND

Messenger Ribonucleic Acid (mRNA) vaccines and methods of treatment thereof have received widespread attention for their potential in fighting a variety of diseases, including infectious diseases and cancers. The translation efficiency and stability of mRNA sequences are particularly important for the design of mRNA sequences.

Methods described in this section are not necessarily methods that have been previously conceived or employed. It should not be assumed that any of the methods described in this section is considered to be the related art just because they are included in this section, unless otherwise indicated expressly. Similarly, the problem mentioned in this section should not be considered to be universally recognized in any related art, unless otherwise indicated expressly.

### SUMMARY

The present disclosure provides a method and apparatus for optimizing an mRNA sequence, an electronic device, a computer-readable storage medium, a computer program product, an mRNA molecule, a pharmaceutical composition, and a use of the mRNA.

According to one aspect of the present disclosure, there provided a method for optimizing an mRNA sequence, comprising obtaining a first mRNA sequence for synthesizing a protein of interest, wherein the first mRNA sequence includes a 5′ untranslated region sequence and a coding region sequence; and adjusting 5′ untranslated region sequence and the coding region sequence with the goal of maximizing a first score of the first mRNA sequence, so as to obtain an optimized second mRNA sequence for synthesizing the protein of interest, where the first score reflects at least one of the following indicators of the first mRNA sequence: translation initiation efficiency, codon adaptation index, and minimum free energy.

According to another aspect of the present disclosure, there provided an apparatus for optimizing an mRNA sequence, comprising an acquisition unit configured to obtain a first mRNA sequence for synthesizing a protein of interest, wherein the first mRNA sequence comprises a 5′ untranslated region sequence and a coding region sequence; and a processing unit configured to adjust 5′ untranslated region sequence and the coding region sequence with the goal of maximizing the first score of the first mRNA sequence, so as to obtain an optimized second mRNA sequence for synthesizing the protein of interest, wherein the first score reflects at least one of the following indicators of the first mRNA sequence: translation initiation efficiency, codon adaptation index, and minimum free energy.

According to another aspect of the present disclosure, there provided an electronic device, including at least one processor; and a memory having a computer program stored thereon, wherein the computer program when executed by the processor causes the processor to perform the foregoing method.

According to another aspect of the present disclosure, there provided a computer-readable storage medium having a computer program stored thereon, wherein the computer program when executed by a processor causes the processor to perform the foregoing method.

According to another aspect of the present disclosure, there provided a computer program product, wherein the computer program product includes a computer program, when executed by a processor, causes the processor to perform the foregoing method.

According to another aspect of the present disclosure, there provided an mRNA molecule, the sequence of which is prepared by the foregoing method.

According to another aspect of the present disclosure, there provided a pharmaceutical composition, and the pharmaceutical composition is composed of the mRNA sequence or molecule prepared by the foregoing method and a pharmaceutically acceptable adjuvant.

According to another aspect of the present disclosure, there provided a use of an mRNA sequence or molecule prepared by the foregoing method or the foregoing pharmaceutical composition in the preparation of a medicine or a vaccine.

It should be understood that the content described in this section is not intended to identify critical or important features of the embodiments of the present disclosure, and is not used to limit the scope of the present disclosure. Other features of the present disclosure will be readily understood with reference to the following description.

### BRIEF DESCRIPTION OF THE DRAWINGS

The accompanying drawings show exemplary embodiments and form a part of the specification, and are used to explain exemplary implementations of the embodiments together with a written description of the specification. The embodiments shown are merely for illustrative purposes and do not limit the scope of the claims. Throughout the accompanying drawings, the same reference numerals denote similar but not necessarily same elements.

FIG. 1 shows a flow chart of a method for optimizing an mRNA sequence according to the embodiments of the present disclosure;

FIG. 2 shows a flow chart of a process for obtaining a 5′ untranslated region sequence of a first mRNA sequence for synthesizing a protein of interest according to the embodiments of the present disclosure;

FIG. 3 shows a flow chart of a process of obtaining a coding region sequence of a first mRNA sequence for synthesizing a protein of interest according to the embodiments of the present disclosure;

FIG. 4 shows a flow chart of the process of adjusting a 5′ untranslated region sequence and a coding region sequence with the goal of maximizing the first score of a first mRNA sequence, so as to obtain an optimized second mRNA sequence for synthesizing a protein of interest according to the embodiments of the present disclosure;

FIG. 5 shows a flow chart of another process of adjusting a 5′ untranslated region sequence and a coding region sequence with the goal of maximizing the first score of a first mRNA sequence, so as to obtain an optimized second mRNA sequence for synthesizing a protein of interest according to the embodiments of the present disclosure;

FIG. 6 shows a flow chart of a process of adjusting a translation initiation region sequence with the goal of maximizing the translation initiation efficiency of a first mRNA sequence, so as to obtain a fourth mRNA sequence according to the embodiments of the present disclosure;

FIG. 7 shows a flow chart of a process of calculating the translation initiation efficiency of each of at least one fifth mRNA sequence according to the embodiments of the present disclosure;

FIG. 8 shows a flow chart of a process of adjusting the coding region main sequence of the fourth mRNA sequence with the goal of maximizing the first score of the fourth mRNA sequence, so as to obtain the second mRNA sequence according to the embodiments of the present disclosure;

FIG. 9 shows a block diagram of a structure of an apparatus for optimizing an mRNA sequence;

FIG. 10 shows the distribution of the mRNA sequence in metric space and the corresponding protein expression level thereof;

FIG. 11 shows the mediation effect of method 100 on the three indicators of TIE, MFE, and CAI;

FIG. 12 shows the comparison of indicators among the wild-type mRNA sequence, the mRNA sequence designed by LinearDesign, the mRNA sequence designed by method 100, and the mRNA sequence designed by a third party.

FIG. 13 shows the results of analyzing the accuracy of TIE indicator using massively parallel translation assay (MPTA) data; and

FIG. 14 shows a block diagram of a structure of an example of electronic device that can be used to implement the embodiments of the present disclosure.

### DETAILED DESCRIPTION OF THE EMBODIMENTS

In the following description, for the purpose of explanation, specific details are set forth to provide an understanding of the present disclosure. However, it will be apparent to those skilled in the art that the present disclosure may be practiced without these details. Furthermore, those skilled in the art will recognize that the embodiments of the present disclosure described below may be implemented in a variety of ways, such as processes, apparatuses, systems, devices, or methods on tangible computer-readable media.

The components or modules shown in the figures are illustrative of embodiments of the present disclosure and are intended to avoid obscuring the present disclosure. It will also be understood that throughout the discussion, components may be described as separate functional units, which may comprise sub-units, but those skilled in the art will recognize that various components, or portions thereof, may be divided into separate components or may be integrated together, including, for example, in a single system or component. It should be noted that the functionality or operations discussed herein can be implemented as components. Components may be implemented in software, hardware, or a combination thereof.

Furthermore, connections between components or systems in the figures are not intended to be limited to direct connections. Instead, data between these components may be modified by intermediate components, reformatted, or otherwise changed. Additionally, more or fewer connections can be used. It should also be noted that the terms “coupling”, “connecting”, “communication coupling”, “interface”, “access” or any derivative thereof shall be understood to comprise direct connection, indirect connection through one or more intermediate devices, and wireless connection. It should also be noted that any communication, such as signals, responses, replies, acknowledgments, messages, queries, etc., may comprise one or more information exchanges.

References in the specification to “one or more embodiments,” “preferred embodiment,” “an embodiment,” “embodiments,” etc., mean that a particular feature, structure, characteristic, or function described in connection with the embodiment is included in at least one embodiment of the present disclosure and may be included in more than one embodiment. Furthermore, the occurrences of the above phrases in various places in the specification may not necessarily all refer to the same embodiment or embodiments.

The use of certain terms in various places in the specification is for the purpose of explanation and should not be construed as limiting. A service, a function, or a resource is not limited to a single service, function, or resource; the use of these terms may refer to a group of related services, functions, or resources, which may be distributed or aggregated. The terms “include,” “comprise,” “has,” and “contain” are to be understood as open-ended terms, and any list below is an example and is not meant to be limited to the items listed. A “layer” can comprise one or more operations. The words “optimal,” “optimize,” “optimization,” and the like refer to improvements in results or processes and do not require that the specified results or processes have reached “optimal” or peak conditions. The terms memory, database, information repository, data store, table, hardware, cache, etc., may be used herein to refer to system components or components into which information can be entered or otherwise recorded.

In one or more embodiments, stopping conditions may comprise: (1) the set number of iterations has been executed; (2) a certain processing time has been reached; (3) convergence (e.g., the difference between consecutive iterations is less than a first threshold); (4) divergence (e.g., performance deterioration); (5) acceptable results have been achieved.

Those skilled in the art should realize: (1) certain steps can be optionally performed; (2) the steps may not be limited to the specific order set forth herein; (3) certain steps can be performed in different orders; (4) some steps can be performed simultaneously.

Any headings used herein are for organizational purposes only and shall not be used to limit the scope of the specification or claims. Each reference/document mentioned in this patent document is incorporated by reference in its entirety.

It should be noted that any experiments and results provided herein are provided by way of illustration and were performed under specific conditions using specific examples. Therefore, neither these experiments nor their results should be used to limit the scope of disclosure of this patent document.

The translation efficiency and stability of mRNA sequences are particularly important for the design of mRNA sequences. Translation efficiency indicates how fast the mRNA sequence can produce proteins, and stability indicates how long the mRNA sequence can continuously translate proteins. Translation efficiency and stability together determine the amount of protein that an mRNA sequence can produce, and ultimately affect the actual effectiveness of an mRNA vaccine, drug, or therapy.

mRNA design methods in related technologies usually focus on designing a single segment in the mRNA, such as 5′ untranslated region or coding region, without considering the interaction between the segments, and cannot finely adjust the translation efficiency and stability of mRNA from an overall perspective.

To address the above problems, embodiments of the present disclosure provide a method for optimizing an mRNA sequence. This method jointly optimizes 5′ untranslated region and the coding region with the goal of maximizing the first score of the mRNA sequence. The first score can reflect at least one indicator among translation initiation efficiency, codon adaptation index, and minimum free energy, enabling targeted optimization of the translation efficiency and stability of the mRNA sequence according to design requirements. This, in turn, optimizes the yield of the final protein of interest, enhancing the overall efficacy of mRNA vaccines and methods of treatment.

According to one or more embodiments of the present disclosure, targeted optimization of the translation efficiency and stability of mRNA is achieved from a holistic perspective by jointly optimizing 5′ untranslated region (UTR) and coding sequence (CDS) of mRNA with the goal of maximizing the first score of mRNA, thereby optimizing the final protein yield and improving the overall efficacy of mRNA vaccines and methods of treatment.

Exemplary embodiments of the present disclosure are described in detail below with reference to the drawings.

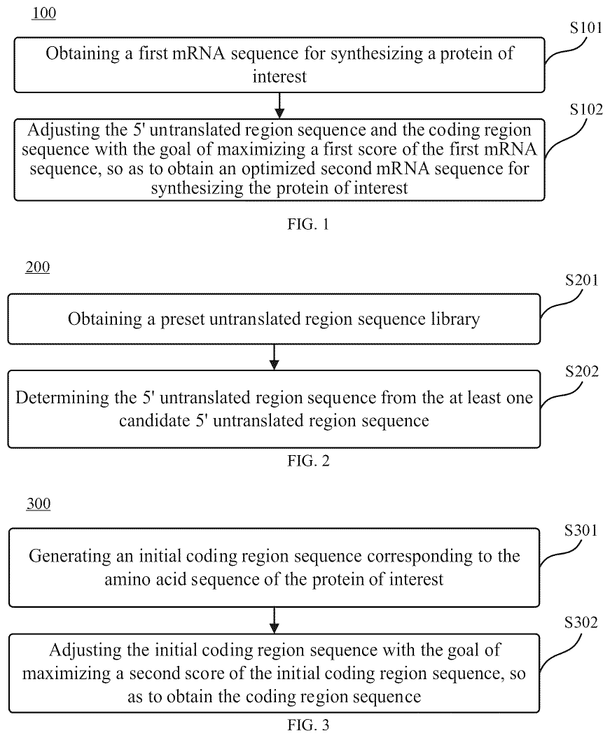

According to one aspect of the present disclosure, there provided a method for optimizing a messenger ribonucleotide (mRNA) sequence. FIG. 1 shows a flow chart of a method 100 for optimizing a messenger ribonucleotide (mRNA) sequence according to an embodiment of the present disclosure. As shown in FIG. 1, the method 100 includes the following steps: step S101: obtaining a first mRNA sequence for synthesizing a protein of interest, wherein the first mRNA sequence includes a 5′ untranslated region sequence and a coding region sequence; and step S102: adjusting 5′ untranslated region sequence and the coding region sequence with the goal of maximizing the first score of the first mRNA sequence, so as to obtain an optimized second mRNA sequence for synthesizing the protein of interest, wherein the first score reflects at least one of the following indicators of the first mRNA sequence: translation initiation efficiency, codon adaptation index, and minimum free energy.

According to an embodiment of the present disclosure, 5′ UTR and CDS are jointly optimized with the goal of maximizing the first score of the mRNA sequence. The first score can reflect at least one indicator among translation initiation efficiency, codon adaptation index, and minimum free energy, enabling targeted optimization of the translation efficiency and stability of the mRNA sequence according to design requirements. This, in turn, optimizes the yield of the final protein of interest, enhancing the overall efficacy of mRNA vaccines and methods of treatment.

In the mRNA sequence, 5′ untranslated region sequence, also known as the 5′ UTR sequence, is located at the 5′ end of the mRNA molecule, starting after 5′ cap structure and ending before the coding region. This segment has the function of regulating translation, meaning that 5′ UTR contains a regulatory element, such as upstream open reading frames (uORFs), suboptimal binding sites (such as GC-rich regions), and regulatory sequences. This segment can affect mRNA stability and translation efficiency. In addition, this segment ensures the stability of the mRNA molecule, with certain sequence elements of this segment helping to protect the mRNA from degradation. This segment can promote ribosome binding and initiate the translation process by ribosome recognition and binding to specific sequences (such as Kozak sequence). In mRNA processing, the signal sequence in 5′ UTR plays a decisive role in mRNA splicing and maturation.

In the mRNA sequence, the coding region sequence, also known as the CDS sequence, is located between 5′ untranslated region and 3′ untranslated region of the mRNA molecule. The coding region sequence contains an open reading frame (ORF), which consists of a series of codons, with each codon corresponding to a specific amino acid, and this sequence is translated into protein in the ribosome. The coding region contains all the genetic information required for protein synthesis, and the coding region usually starts with a start codon (such as AUG) and ends with a stop codon (such as UAA, UAG or UGA).

The 5′ untranslated region and the coding region are critical for protein synthesis. The 5′ untranslated region is involved in regulating the stability and translation efficiency of mRNA, while the coding region directly determines the amino acid sequence of the protein. Through joint optimization of 5′ untranslated region and the coding region, the translation efficiency and stability of mRNA can be improved as a whole, thereby increasing the final yield of the protein of interest.

In the embodiment of the present disclosure, in step S102, 5′ untranslated region sequence and the coding region sequence are jointly adjusted with the goal of maximizing the first score of the first mRNA sequence, so as to obtain an optimized second mRNA sequence. The first score can reflect at least one of the following indicators of the first mRNA sequence: translation initiation efficiency (TIE), codon adaptation index (CAI), and minimum free energy (MFE). That is, the first score is calculated based on at least one of the three indicators: translation initiation efficiency, codon adaptation index, and minimum free energy of the first mRNA sequence.

Translation initiation efficiency (TIE) is used to measure the translation initiation efficiency of ribosomes on mRNA molecules, thereby measuring the translation efficiency of the mRNA sequence. The larger the value of TIE, the faster the translation process starts, and the higher the translation efficiency of the mRNA sequence. In the case where the first score is calculated based on the TIE, step S102 can achieve targeted optimization of the TIE of the mRNA sequence, thereby improving the overall efficiency of protein synthesis and ensuring that the mRNA can produce a robust and rapid response when entering the cell.

The codon adaptation index (CAI) measures how consistent the codons in an mRNA sequence are with the most commonly used codons in the host cell. The larger the value of CAI, the closer the codons used in the mRNA sequence are to the codons of highly expressed genes in the host cell, which may lead to higher translation efficiency. In the case where the first score is calculated based on CAI, step S102 can achieve targeted optimization of the CAI of the mRNA sequence to ensure that the mRNA uses codons preferred by the translation mechanism of the host, thereby improving the rate and accuracy of protein synthesis.

Minimum free energy (MFE) is used to measure the energy state of mRNA molecules when forming secondary structures, thereby measuring the structural stability of mRNA molecules. The smaller the value of MFE, the more stable the structure of the mRNA. Stable mRNA structure helps protect mRNA from degradation, thereby improving its stability and half-life within cells. In the case where the first score is calculated based on MFE, step S102 can achieve targeted optimization of the MFE of the mRNA sequence, thereby improving the stability of the mRNA, protecting the mRNA from degradation, and enhancing its survival time in the cellular environment. However, an overly stable structure may hinder ribosome binding and translation initiation. Therefore, MFE needs to be balanced with TIE and CAI to achieve optimal performance of mRNA.

It can be understood that since TIE and CAI are positively correlated with the translation efficiency of mRNA, and MFE is negatively correlated with the stability of mRNA, in order to optimize the translation efficiency and stability of the mRNA sequence, the first score can be set to be positively correlated with TIE and CAI, and negatively correlated with MFE.

In some embodiments, the first score S can be calculated according to the following formula (1):

$$S = {λ}_{TIE} * TIE + {λ}_{CAI} * CAI - {λ}_{MFE} * MFE \qquad ( 1 )$$

among them, λ_{TIE}, λ_{CAI} and λ_{MFE} are the weights of TIE, CAI, and MFE indicators respectively. The values of λ_{TIE}, λ_{CAI} and λ_{MFE} can be set according to the design requirements of the mRNA to achieve the balance and flexible regulation of the three indicators of TIE, CAI, and MFE, allowing the generated mRNA sequence to have the desired characteristics.

In some embodiments, the weight of a certain indicator in formula (1) can be set to a fixed value (for example, 1), and the balance among the three indicators can be achieved by adjusting the weights of the other two indicators. For example, the weight of the MFE indicator can be set to 1 and the weights of TIE and CAI are adjusted to achieve a balance among TIE, CAI, and MFE. In this embodiment, formula (1) is simplified to the following formula (2):

$$S = {λ}_{TIE} * TIE + {λ}_{CAI} * CAI - MFE \qquad ( 2 )$$

In some embodiments, the first score S can be calculated according to the following formula (3):

$$S = {λ}_{TIE} * L * log ( TIE ) + {λ}_{CAI} * L * log ( CAI ) - MFE \qquad ( 3 )$$

In the above formula (3), L is the number of codons included in the coding region sequence. By introducing L into the TIE term and CAI term, the values of the TIE term, CAI term, and MFE term in formula (3) can be of similar magnitude, thus facilitating the realization of balance and flexible regulation of the three indicators of TIE, CAI, and MFE. By performing logarithmic transformation on TIE and CAI (expressed as log(TIE) and log(CAI) respectively), the multiplication operation between the internal factors in calculating TIE and CAI can be converted into addition operation, thereby simplifying the calculation.

It should be noted that mRNA further comprises other component segments in addition to 5′ untranslated region and the coding region, such as 5′ cap structure, 3′ untranslated region, and poly(A) tail. Embodiments of the present disclosure jointly optimize the 5′ untranslated region and the coding region. Although other segments in the mRNA are not optimized (the preset segments can be used directly), they may be involved in the calculation of the first score. For example, 3′ untranslated region may be related to the value of TIE (e.g., the structural features of 3′ untranslated region are taken into account when calculating TIE), and therefore will have an impact on the first score S.

By jointly optimizing 5′ untranslated region and coding region using three indicators: TIE, CAI, and MFE, the optimized second mRNA sequence can balance the three key aspects of translation initiation efficiency, translation elongation efficiency (corresponding to CAI) and stability, thereby optimizing the final protein yield.

The TIE of an mRNA sequence can be calculated, for example, by the translation initiation efficiency prediction model described below. The CAI of an mRNA sequence can be obtained, for example, by comparing the codon usage of the mRNA sequence with the preset codon usage of highly expressed genes. The MFE of an mRNA sequence can be calculated, for example, by algorithms such as the thermodynamic perturbation method and thermodynamic calculus method.

In some embodiments, for step S101, each component segment of the first mRNA sequence can be obtained separately, and then the respective component segments can be spliced to obtain the first mRNA. Specifically, 5′ untranslated region sequence of the first mRNA sequence can be obtained through the following process 200. The coding region sequence of the first mRNA sequence can be obtained through the following process 300. Other component segments in the first mRNA sequence, such as 3′ untranslated region sequence, can adopt preset values.

FIG. 2 shows a flow chart of a process 200 for obtaining a 5′ untranslated region sequence of a first mRNA sequence for synthesizing a protein of interest according to an embodiment of the present disclosure. The process 200 may be used to implement step S101 in the foregoing method 100. In some embodiments, as shown in FIG. 2, process 200 may include: step S201, obtaining a preset untranslated region sequence library, wherein the untranslated region sequence library comprises at least one candidate 5′ untranslated region sequence, and each candidate 5′ untranslated region sequence in the at least one candidate 5′ untranslated region sequence enables gene expression; and step S202, determining 5′ untranslated region sequence included in the first mRNA sequence from at least one candidate 5′ untranslated region sequence.

According to the above embodiments, selecting a known 5′ untranslated region sequence that can achieve gene expression as the initial value of 5′ untranslated region sequence in the mRNA sequence can ensure the quality of 5′ untranslated region sequence, providing better samples for subsequent further optimization.

In some embodiments, in step S201, in order to ensure that 5′ untranslated region sequence in the first mRNA sequence is a sequence that can be normally expressed, a untranslated region sequence library can be constructed based on known mRNA databases, such as UTRdb, NCBI (National Center for Biotechnology Information), UTRsite, EMBL (European Molecular Biology Laboratory Database), ENSEMBL and other databases. The candidate 5′ untranslated region sequence in the untranslated region sequence library can be a natural sequence from the aforementioned mRNA databases or a sequence obtained through artificial optimization. The selection range of 5′ untranslated region sequences can be expanded by constructing an untranslated region sequence library to provide better samples for subsequent optimization.

In some embodiments, in step S202, a 5′ untranslated region sequence is selected from the untranslated region sequence library constructed in S201 as 5′ untranslated region sequence in the first mRNA sequence. By selecting a 5′ untranslated region sequence from the untranslated region sequence library, it can be ensured that the selected 5′ untranslated region sequence has normal expression ability and will not have an adverse effect on subsequent optimization.

FIG. 3 shows a flow chart of a process 300 of obtaining a coding region sequence of a first mRNA sequence for synthesizing a protein of interest according to an embodiment of the present disclosure. The process 300 may be used to implement step S101 in the foregoing method 100. In some embodiments, as shown in FIG. 3, process 300 may include: step S301: generating an initial coding region sequence corresponding to the amino acid sequence of the protein of interest; and step S302: adjusting the initial coding region sequence with the goal of maximizing the second score of the initial coding region sequence, so as to obtain the coding region sequence, wherein the second score reflects the codon adaptation index and/or minimum free energy of the initial coding region sequence.

According to the above embodiments, the initial coding region sequence is adjusted with the goal of maximizing the second score, so that the resulting coding region sequence can be translated into the protein of interest while achieving a balance between translation efficiency and stability, thereby providing a better sample for subsequent further optimization. In embodiments of the present disclosure, the protein of interest may be any given protein. Since the protein of interest is determined, its amino acid sequence can be obtained.

In some embodiments, in step S301, the amino acid sequence of the protein of interest can be obtained based on known information or through conventional technical means including but not limited to: gene cloning and sequencing, transcriptome sequencing, protein sequencing, computational prediction, yeast two-hybrid system and protein chip. Through the corresponding rules of amino acids and codons, the codon corresponding to each amino acid in the protein of interest can be obtained, and then the codon corresponding to each amino acid of the protein of interest can be spliced to obtain the initial coding region sequence. Through the foregoing method, an accurate initial coding region sequence that can be translated into the protein of interest can be provided for the first mRNA sequence, thereby ensuring the expression ability of the final generated second mRNA sequence after optimization.

In some embodiments, in step S302, the initial coding region sequence is adjusted with the goal of maximizing the second score of the initial coding region sequence in step S301, so as to obtain an optimized coding region sequence as a component of the first mRNA sequence. The second score can reflect the codon adaptation index and/or minimum free energy of the initial coding region sequence. That is, the second score is calculated based on the codon adaptation index and/or minimum free energy of the initial coding region sequence.

In some embodiments, the second score S′ can be calculated according to the following formula (4):

$${S}^{′} = - {λ}_{MFE} * MFE + {λ}_{CAI} * CAI \qquad ( 4 )$$

among them, λ_{MFE} and λ_{CAI} are the weights of the MFE and CAI indicators, respectively. The values of λ_{MFE} and λ_{CAI} can be set according to needs, thereby achieving a balance and flexible regulation of MFE and CAI indicators, so that the generated coding region sequence has the required characteristics.

In some embodiments, the weight of a certain indicator in formula (4) can be set to a fixed value (for example, 1), and the balance between the two indicators can be achieved by adjusting the weight of the other indicator. For example, the weight of the MFE indicator can be set to 1 and the weight of CAI is adjusted to achieve a balance between MFE and CAI. In this embodiment, formula (4) is simplified to the following formula (5):

$${S}^{′} = - MFE + {λ}_{CAI} * CAI \qquad ( 5 )$$

In some embodiments, the second score S′ can be calculated according to the following formula (6):

$${S}^{′} = - MFE + {λ}_{CAI} * L * log ( CAI ) \qquad ( 6 )$$

In the above formula, L is the number of codons included in the coding region sequence. By introducing L into the CAI term, the values of the CAI term and MFE term in formula (6) can be of similar magnitude, thus facilitating the realization of balance and flexible regulation of the three indicators of MFE and CAI. By performing logarithmic transformation on CAI (expressed as log(CAI)), the multiplication operation between the internal factors in calculating CAI can be converted into addition operation, thereby simplifying the calculation.

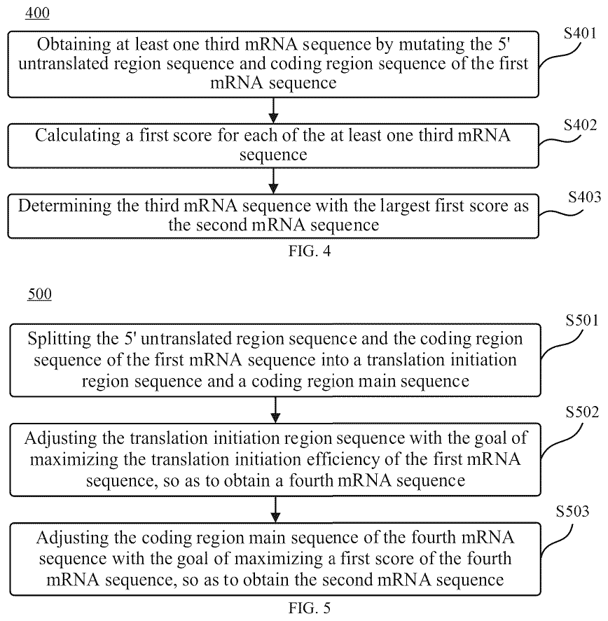

FIG. 4 shows a flow chart of the process 400 of adjusting a 5′ untranslated region sequence and a coding region sequence with the goal of maximizing the first score of a first mRNA sequence, so as to obtain an optimized second mRNA sequence for synthesizing a protein of interest according to an embodiment of the present disclosure. The process 400 may be used to implement step S102 in the foregoing method 100. In some embodiments, as shown in FIG. 4, process 400 may include: step S401: obtaining at least one third mRNA sequence by mutating the 5′ untranslated region sequence and coding region sequence of the first mRNA sequence; step S402: calculating a first score for each of the at least one third mRNA sequence; and step S403: determining the third mRNA sequence with the highest first score as the second mRNA sequence.

According to the above embodiments, an efficient and stable second mRNA sequence can be obtained more quickly by simultaneously performing mutation adjustments on the 5′ untranslated region sequence and the coding region sequence of the first mRNA sequence.

In some embodiments, in step S401, mutations are performed simultaneously on the 5′ untranslated region sequence and the coding region sequence of the first mRNA sequence to obtain at least one third mRNA sequence. The third mRNA sequence is obtained by randomly changing the nucleotides of 5′ untranslated region sequence and the coding region sequence in the first mRNA. Specifically, 5′ untranslated region sequence and the coding region sequence can be mutated one or more times (that is, a randomly selected nucleotide at a certain position is substituted with another nucleotide) as a whole, and each mutation results in a third mRNA sequence. New sequences can be explored and obtained by mutating 5′ untranslated region sequence and coding region sequence, providing more sequence samples for subsequent screening and optimization.

In some embodiments, in step S402, the calculation method of the first score as shown above is applied to each third mRNA sequence, such as formula (1)-formula (3), to calculate the first score of the third mRNA sequence.

In some embodiments, in step S403, at least one third mRNA sequence obtained by mutation is screened according to the first score, and the third mRNA sequence with the highest first score is determined as the optimized second mRNA sequence. The second mRNA sequence having the highest first score means that this sequence has the best overall performance among numerous third mRNA sequences and can achieve a balance of translation initiation efficiency, translation elongation efficiency (corresponding to CAI), and stability.

In some embodiments, steps S401-S403 can be executed multiple times in a loop manner, and the second mRNA sequence obtained in the current loop (i.e., the optimization result of the current loop) can be used as the first mRNA sequence of the next loop (i.e., the starting point for optimization in the next loop), thereby achieving iterative optimization of the mRNA sequence and obtaining the optimal second mRNA sequence. The loop of steps S401-S403 continues until the preset termination condition is satisfied. The termination condition may include, for example, the number of loops reaching a predetermined loop number threshold, the first score of the second mRNA sequence reaching a predetermined first score threshold, or the first score of the second mRNA sequence no longer significantly improving (i.e., the first score converges), etc. After the loop is terminated, the second mRNA sequence obtained in the last loop is used as the final mRNA optimization result.

The above process 400 can be understood as an evolutionary algorithm. In this algorithm, the first score is the fitness value of each third mRNA sequence obtained by mutation.

FIG. 5 shows a flow chart of another process 500 of adjusting a 5′ untranslated region sequence and a coding region sequence with the goal of maximizing the first score of a first mRNA sequence, so as to obtain an optimized second mRNA sequence for synthesizing a protein of interest according to an embodiment of the present disclosure. The process 500 may be used to implement step S102 in the foregoing method 100. In some embodiments, as shown in FIG. 5, process 500 may include: step S501: splitting 5′ untranslated region sequence and the coding region sequence of the first mRNA sequence are into a translation initiation region sequence and a coding region main sequence, wherein the translation initiation region sequence comprises at least 5′ untranslated region sequence, and the coding region main sequence comprises nucleotides in the coding region sequence that are not included in the translation initiation region sequence; step S502: adjusting the translation initiation region sequence with the goal of maximizing the translation initiation efficiency of the first mRNA sequence, so as to obtain a fourth mRNA sequence; and step S503: adjusting the coding region main sequence of the fourth mRNA sequence with the goal of maximizing the first score of the fourth mRNA sequence, so as to obtain the second mRNA sequence.

According to the above embodiments, based on the impact of each component segment of the mRNA on the protein translation process, 5′ untranslated region sequence and the coding region sequence are split into two parts: the translation initiation region sequence and the coding region main sequence. These two parts are successively optimized according to different optimization goals, thereby achieving more precise and targeted optimization of the translation efficiency and stability of mRNA.

In some embodiments, in step S501, 5′ untranslated region sequence and the coding region sequence of the first mRNA are adjusted, and 5′ untranslated region sequence of the first mRNA and a preset number of nucleotides in the coding region sequence close to 5′ untranslated region sequence form a translation initiation region sequence. In some embodiments, the preset number is preferably 30. Because during the translation process, ribosomes need to carry codons for translation, ribosomes occupy approximately 30 nucleotides in length on the mRNA. The residence time of ribosomes in the leader region of the coding region may affect the assembly and translation initiation of subsequent ribosomes, thereby affecting the translation efficiency of mRNA. Therefore, when setting the translation initiation region sequence, it is necessary to consider the position of the ribosome occupying the mRNA, and divide the first 30 nucleotides of the coding region sequence into the translation initiation region sequence.

As described above, 5′ untranslated region sequence and a preset number (for example, 30) of nucleotides of the coding region sequence close to 5′ untranslated region sequence affects ribosome assembly and translation initiation. The overall optimization of the translation initiation region sequence composed of the two parts can improve the translation initiation efficiency in a targeted manner, thereby enhancing the translation efficiency of the mRNA sequence.

In some embodiments, after obtaining the translation initiation region sequence, certain preprocessing can be performed on the translation initiation region sequence, and the preprocessed translation initiation region sequence can be optimized. Preprocessing of the translation initiation region sequence comprises, for example, identifying the −3 position at the end of 5′ UTR and ensuring that this position is a purine (A or G) so that it conforms to Kozak sequence characteristics, which helps to improve the efficiency of translation initiation. The preprocessing operation of the translation initiation region sequence further comprises, for example, analyzing 5′ UTR region and identifying all possible upstream initiation codons (uAUG). For each identified uAUG, any nucleotide in the AUG is replaced with another type of nucleotide to prevent it from serving as a translation initiation site, so that the translation efficiency is further improved, and the initiation site of the translation process is ensured to be accurate, avoiding translation misalignment that could prevent the production of the protein of interest.

In some embodiments, in step S502, the translation initiation region sequence in the first mRNA sequence is adjusted with the goal of maximizing the translation initiation efficiency of the first mRNA sequence, so as to obtain a fourth mRNA sequence. It can be understood that there are differences in the translation initiation region sequences of the fourth mRNA sequence and the first mRNA sequence, but the coding region main sequences of the two are the same. According to this embodiment, the fourth mRNA sequence has maximized translation initiation efficiency, which provides a solid foundation for subsequent optimization of the coding region main sequence.

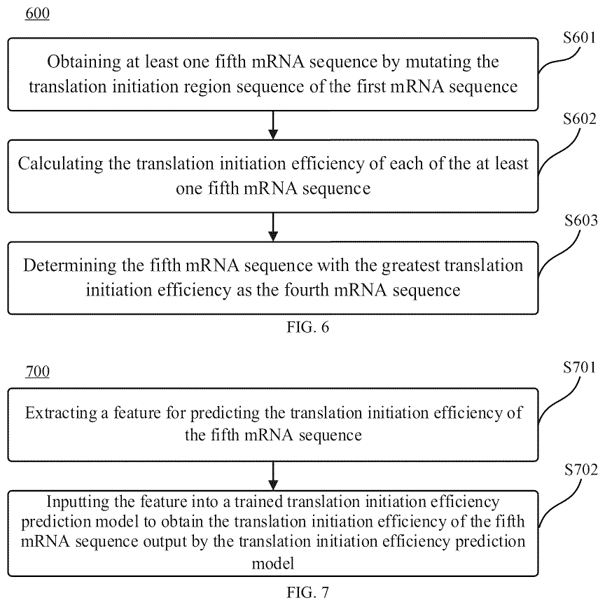

FIG. 6 shows a flow chart of a process 600 of adjusting a translation initiation region sequence with the goal of maximizing the translation initiation efficiency of a first mRNA sequence, so as to obtain a fourth mRNA sequence according to an embodiment of the present disclosure. The process 600 may be used to implement the above step S502. In some embodiments, as shown in FIG. 6, process 600 may include: step S601: obtaining at least one fifth mRNA sequence by mutating the translation initiation region sequence of the first mRNA sequence; step S602: calculating the translation initiation efficiency of each of the at least one fifth mRNA sequence; and step S603: determining the fifth mRNA sequence with the greatest translation initiation efficiency as the fourth mRNA sequence.

According to the above embodiments, the translation initiation efficiency of the fourth mRNA can be effectively improved, thereby ensuring the translation efficiency of the finally generated second mRNA.

In some embodiments, in step S601, the number of translation initiation region sequences can be enriched by performing a plurality of mutations on the translation initiation region sequence of the first mRNA sequence, thereby obtaining a plurality of fifth mRNA sequences. Through the foregoing method, the amount of samples to be optimized can be increased, providing a rich sample basis for subsequent screening of the fifth mRNA sequence with the highest translation initiation efficiency.

In some embodiments, in step S602, the translation initiation efficiency of each fifth mRNA sequence is calculated.

FIG. 7 shows a flow chart of a process 700 of calculating the translation initiation efficiency of each of at least one fifth mRNA sequence according to an embodiment of the present disclosure. The process 700 may be used to implement step S602 in the foregoing method 600. In some embodiments, as shown in FIG. 7, process 700 may include: the following steps are performed for each of the at least one fifth mRNA sequence: step S701: extracting a feature for predicting the translation initiation efficiency of the fifth mRNA sequence; and step S702: inputting the feature into a trained translation initiation efficiency prediction model to obtain the translation initiation efficiency of the fifth mRNA sequence output by the translation initiation efficiency prediction model.

Translation initiation efficiency may be affected by a plurality of factors. According to the above embodiments, the accuracy and generalization of the translation initiation efficiency evaluation can be improved by extracting the features of the fifth mRNA sequence and analyzing these features using a trained translation initiation efficiency prediction model to obtain the translation initiation efficiency of the fifth mRNA sequence.

In some embodiments, in step S701, one or more features of the fifth mRNA sequence are extracted as input to the translation initiation efficiency prediction model. In some embodiments, the features used to predict the translation initiation efficiency of the fifth mRNA sequence comprise at least one of the following: Structural compactness of the translation initiation region (TIR_ddG_pNT), whole structural compactness (whole_MFE_pNT), Kozak sequence feature (prime_m3), upstream initiation codon (uAUG) and upstream open reading frame (uORF) sequence feature, and ribosome residence time in the CDS leader region (CDS_leader_DT).

According to the above embodiments, the sequence characteristics and structural features of the translation initiation region of the fifth mRNA sequence can be flexibly and comprehensively obtained by flexibly selecting a feature combination for predicting translation initiation efficiency, thereby more accurately predicting the translation initiation efficiency of the translation initiation region of the fifth mRNA sequence.

The structural compactness of the translation initiation region (TIR_ddG_pNT) represents the free energy change of the secondary structure of the translation initiation region (including 5′ UTR and 5′ leader sequence of the CDS) before and after unfolding. A lower free energy change indicates a more compact structure and is generally associated with a lower TIE.

The whole structural compactness (whole_MFE_pNT) feature measures the minimum free energy (MFE) of the entire mRNA sequence (including 5′ UTR, CDS, and 3′ UTR), and normalized to sequence length. A higher normalized MFE indicates that the whole structure is less stable, which generally correlates positively with TIE.

Kozak sequence feature (prime_m3): The presence of a purine (A/G) at the −3 position of 5′ UTR is a hallmark of Kozak sequences and can enhance translation initiation. This feature is positively correlated with TIE.

uAUG and uORF sequence features include:

In-frame upstream open reading frame (in_frame_uORF): The presence of upstream open reading frames (uORFs) in-frame with the main open reading frame (ORF) can inhibit downstream translation and negatively affect TIE.

Start codon out of reading frame (out_frame_uAUG): The start codon is located upstream of the main open reading frame (ORF), out of the reading frame and negatively correlated with TIE.

The ribosome dwell time of CDS leader region (CDS_leader_Dwell Time) feature measures the residence time of ribosomes in 5′ leader region of CDS. Since the ribosome occupies approximately 30 nucleotides on the mRNA, a longer residence time may interfere with subsequent ribosome assembly and translation initiation, and is therefore negatively correlated with TIE.

The sequence features and structural features of the translation initiation region of the fifth mRNA can be obtained comprehensively and accurately by evaluating the above features, thereby more accurately calculating the translation initiation efficiency.

In some embodiments, in step S702, the translation initiation efficiency of the fifth mRNA sequence output by the translation initiation efficiency prediction model can be obtained by inputting the features obtained in step S701 into the trained translation initiation efficiency prediction model.

The translation initiation efficiency prediction model can be any machine learning model, including but not limited to regression models, decision tree models, random forest models, neural network models, etc. The translation initiation efficiency prediction model can be trained and obtained using sequence features labeled with translation initiation efficiency labels as samples.

In some embodiments, a ridge regression model can be used as a translation initiation efficiency prediction model. The ridge regression model can be used to predict logarithmically transformed TIE (i.e., log(TIE)). This model can handle multicollinearity between features and can prevent overfitting through regularization. The training data for the model can be selected from the multimer analysis data of eGFP and the ribosome analysis data of the human genome, which data can be selected from, for example, the National Genomics Data Center of China. These data sets provide comprehensive insights into the dynamics of mRNA translation. At the same time, the above features for each input are scaled to ensure consistency and improve model performance, and a ridge regression model is built using the above features as predictor variables. Ridge regression introduces a penalty term that is proportional to the square of the coefficient size, and the penalty term avoids overreliance on any single feature. The model is trained on the collected data sets and the performance of the model is evaluated using standard metrics including mean square error (MSE) and R² score as a post-loss function of the model. Cross-validation was used to evaluate the robustness of the model and fine-tune the model's hyperparameters.

Step S603 can be performed after obtaining the translation initiation efficiency of each fifth mRNA sequence through step S602. In step S603, the fifth mRNA sequence with the highest translation initiation efficiency is selected as the fourth mRNA sequence.

In some embodiments, steps S601-S603 can be executed multiple times in a loop manner, and the fourth mRNA sequence obtained in the current loop (i.e., the optimization result of the current loop) can be used as the first mRNA sequence of the next loop (i.e., the starting point for optimization in the next loop), thereby achieving iterative optimization of the mRNA sequence and obtaining the optimal fourth mRNA sequence. The loop of steps S601-S603 continues until the preset termination condition is satisfied. The termination condition may include, for example, the number of loops reaching a predetermined loop number threshold, the translation initiation efficiency of the fourth mRNA sequence reaching a predetermined translation initiation efficiency threshold, or the translation initiation efficiency of the fourth mRNA sequence no longer significantly improving (i.e., the translation initiation efficiency converges), etc. After the loop is terminated, the fourth mRNA sequence obtained in the last loop is used as the optimization result for the translation initiation region sequence.

In some embodiments, process 600 may be understood as an evolutionary algorithm. The specific operation of this algorithm is as follows:

Designing the initial population: the translation initiation region sequence of the first mRNA sequence is mutated to construct an initial population composed of a plurality of mRNA sequences.

Defining the fitness function: the performance of each sequence variant is evaluated using translation initiation efficiency (TIE) as the fitness function.

Iterating the optimization process: the sequence population is iteratively optimized by simulating the process of natural selection and applying mutation and selection operations.

Mutation: the nucleotides in the sequence are randomly changed to explore new sequence space so as to obtain a plurality of fifth mRNA sequences.

Selection: Based on the TIE evaluation results of each fifth mRNA sequence, the sequence with the highest TIE is selected as the current optimal fourth mRNA sequence for the next generation of iteration.

Termination condition: Iterations are stopped when a predetermined number of iterations is reached or when sequence performance no longer improves significantly.

In some embodiments, in step S503, based on the fourth mRNA sequence that has been optimized in the translation initiation region obtained in step S502, the coding region main sequence of the fourth mRNA sequence is adjusted with the goal of maximizing the first score of the fourth mRNA sequence, so as to obtain the optimized second mRNA sequence.

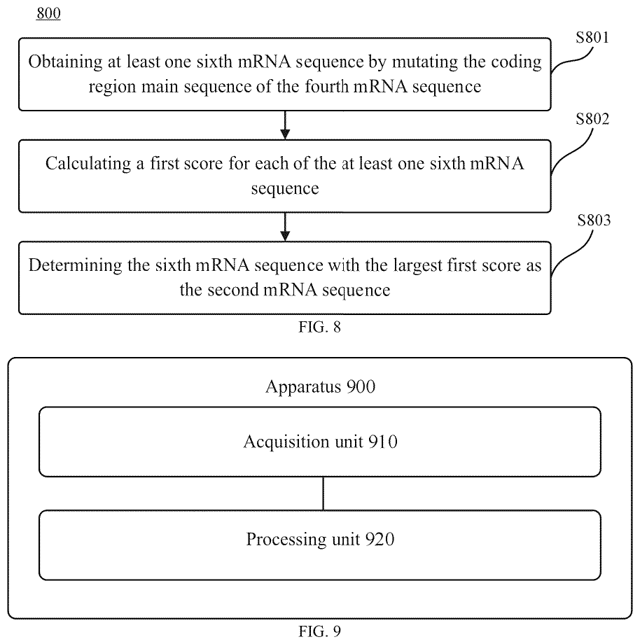

FIG. 8 shows a flow chart of the process 800 of adjusting the coding region main sequence of the fourth mRNA sequence with the goal of maximizing the first score of the fourth mRNA sequence, so as to obtain the second mRNA sequence according to an embodiment of the present disclosure. The process 800 may be used to implement step S503 in the foregoing method 500. In some embodiments, as shown in FIG. 8, process 800 may include: step S801: obtaining at least one sixth mRNA sequence by mutating the coding region main sequence of the fourth mRNA sequence; step S802: calculating a first score for each of the at least one sixth mRNA sequence; and step S803: determining the sixth mRNA sequence with the highest first score as the second mRNA sequence.

According to the above embodiments, the first score can reflect at least one indicator among translation initiation efficiency, codon adaptation index, and minimum free energy, enabling targeted optimization of the translation efficiency and stability of the mRNA sequence according to design requirements, especially the base structure pairing in the translation initiation region is optimized and improved. This, in turn, optimizes the yield of the protein of interest, enhancing the overall efficacy of mRNA vaccines and methods of treatment.

In some embodiments, in step S801, a plurality of mutations are performed on the coding region main sequence of the fourth mRNA sequence, which can enrich the number of the coding region main sequences, thereby obtaining a plurality of sixth mRNA sequences. Through the foregoing method, the amount of samples to be optimized can be increased, providing a sample basis for subsequent screening of the sixth mRNA with the highest first score.

In some embodiments, in step S802, a first score for each sixth mRNA is calculated. In this process, the calculation formulas (1)-(3) for the first score as shown above can be applied to calculate the first score corresponding to each sixth mRNA.

In some embodiments, in step S803, the sixth mRNA sequence with the highest score is selected from the sixth mRNA sequences that have been scored in step S802 as the optimized second mRNA sequence. The second mRNA sequence having the highest first score means that this sequence has the best overall performance among numerous sixth mRNA sequences and can achieve achieves a balance among three key factors: translation initiation efficiency, translation elongation efficiency (corresponding to CAI), and stability.

In some embodiments, in step S803, the sixth mRNA sequence is determined as the optimized second mRNA sequence in response to the codon adaptation index of the sixth mRNA sequence with the highest first score being greater than a threshold, wherein the threshold is determined based on the codon adaptation index of the initial first mRNA sequence (i.e., the first mRNA sequence obtained through step S101). For example, the threshold can be set to the codon adaptation index of the initial first mRNA sequence. According to this embodiment, it can ensure that the optimized second mRNA sequence has a translation expression ability no lower than that of the initial first mRNA sequence.

In some embodiments, the current optimization result can be discarded in response to the codon adaptation index of the sixth mRNA sequence with the highest first score being less than or equal to the threshold, meaning the sixth mRNA sequence will not be used as the optimized second mRNA sequence. Instead, steps S801-S803 are re-executed until an optimization result with a codon adaptation index greater than the threshold is obtained. In some embodiments, steps S801-S803 can be executed multiple times in a loop manner, and the second mRNA sequence obtained in the current loop (i.e., the optimization result of the current loop) can be used as the fourth mRNA sequence of the next loop (i.e., the starting point for optimization in the next loop), thereby achieving iterative optimization of the mRNA sequence and obtaining the optimal second mRNA sequence. The loop of steps S801-S803 continues until the preset termination condition is satisfied. The termination condition may include, for example, the number of loops reaching a predetermined loop number threshold, the first score of the second mRNA sequence reaching a predetermined first score threshold, or the first score of the second mRNA sequence no longer significantly improving (i.e., the first score converges), etc. After the loop is terminated, the second mRNA sequence obtained in the last loop is used as the final mRNA optimization result. In some embodiments, steps S801-S803 can be regarded as applying an evolutionary algorithm to operate, and the operation is specifically as follows:

Designing the initial population: the coding region main sequence of the fourth mRNA sequence is mutated to construct an initial population composed of a plurality of mRNAs.

Defining the fitness function: the performance of each sequence variant is evaluated using the first score as the fitness function.

Iterating the optimization process: the sequence population is iteratively optimized by simulating the process of natural selection and applying mutation and selection operations.

Mutation: Mutation is performed on the position in the coding region main sequence that is paired with the translation initiation region, exploring sequence variants that may improve translation initiation efficiency and/or reduce minimum free energy to obtain a plurality of sixth mRNA sequences.

Selection: Based on the evaluation results of the first score of each sixth mRNA sequence, the sequence with the highest first score is selected as the current optimal second mRNA sequence for the next generation of iteration.

Termination condition: Iterations are stopped when a predetermined number of iterations is reached or when the first score of the second mRNA sequence is no longer significantly improved.

According to embodiments of the present disclosure, there is further provided an apparatus for designing messenger ribonucleotide (mRNA) sequences. FIG. 9 shows a block diagram of a structure of a training apparatus for a neural network model that predicts the impact of mutations on protein stability according to an embodiment of the present disclosure. As shown in FIG. 9, the apparatus 900 includes: an acquisition unit 910 configured to obtain the first mRNA sequence for synthesizing the protein of interest; and a processing unit 920 configured to adjust the 5′ untranslated region sequence and the coding region sequence with the goal of maximizing the first score of the first mRNA sequence, so as to obtain an optimized second mRNA sequence for synthesizing the protein of interest.

It can be understood that the operations of the units 910 to 920 in apparatus 900 may refer to the above description of steps S101 to S102 in method 100. Details are not described herein again.

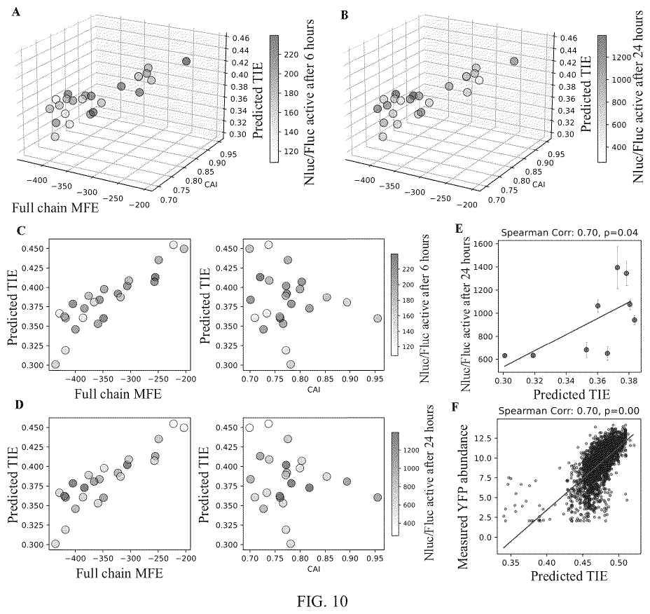

In an embodiment, the differences in actual protein yields of samples from various regions within the metric space are examined to assess the guiding value of the first scoring formula in method 100. The mRNA sequence and expression data come from the article “Kathrin Leppek, Gun Woo Byeon, Wipapat Kladwang, Hannah K Wayment-Steele, Craig H Kerr, Adele F Xu, Do Soon Kim, Ved V Topkar, Christian Choe, Daphna Rothschild, et al. Combinatorial optimization of mRNA structure, stability, and translation for RNA-based therapeutics. Nature communications, 13(1): 1536, 2022”. In this article, the expression levels of Nluc reporter genes with different CDS sequences were measured through the Nluc/Fluc reporter gene activity ratio. As shown in panels A and B in FIG. 10, panel A shows the expression level of each sample after 6 hours, and panel B shows the expression level of each sample after 24 hours, where the darker the color of the dot, the higher the expression level. As shown in the distributions in panels A and B, samples with the highest protein expression are mainly located in regions with low MFE (<−350 kcal/mol), high CAI (>0.75), and moderate TIE (0.35-0.42). As shown in panels C and D, which are two-dimensional distribution graphs of panels A and B respectively, this pattern can also be observed in panels C and D. Samples with predicted too low TIE showed a distinct disadvantage in expression levels at both time points, indicating that adequate translation efficiency is necessary for effective protein yield. There are also differences in the expression level distribution patterns at 6 hours and 24 hours. Specifically, samples with the highest expression levels at 6 hours tend to have higher TIE, while samples with the highest expression levels at 24 hours tend to have lower MFE. This is consistent with the general principle that short-term expression levels are more affected by translation efficiency, whereas long-term expression levels are more affected by mRNA stability.

However, the samples with the highest TIE did not exhibit high protein expression levels in this distribution. This may be because these samples also have relatively high MFE values, resulting in reduced stability and thus affecting their sustained expression ability. The disadvantages between TIE and MFE as optimization targets are understandable, as the reduction of MFE increases the compactness of the mRNA structure, thereby creating a barrier for ribosomes and other translation factors to bind to the mRNA. As shown in panels E and F, panel E is a scatter plot showing the correlation between TIE and Nluc/Fluc activity over 24 hours for samples selected according to the criteria of MFE<−350 kcal/mol and CAI>0.75. Panel F is a scatter plot showing the correlation between TIE and the abundance of YFP expressed in yeast over 24 hours. In panels E and F, when filtering out samples with MFE that is too high (>350 kcal/mol) and CAI that is too low (<0.75), a more pronounced positive correlation between TIE and protein expression levels can be observed. Specifically, the Spearman correlation between TIE and Nluc/Fluc activity ratio reached 0.70 (p<0.05) in the 24-hour expression data. Therefore, better protein yields may be achieved by optimizing TIE while ensuring relatively optimal values of MFE and CAI.

In an embodiment, method 100 introduces two custom parameters, λ_{TIE} and λ_{CAI}, to balance the relative weights of the three optimization objectives, namely TIE, CAI, and MFE. The parameters λ_{CAI} and λ_{TIE} control the weights of CAI and TIE respectively in the optimization process. To enhance the convenience of indicator adjustment, the optimization algorithm ensures that the CAI indicator of the sequence of interest is not affected by the λ_{TIE} value. This means that once λ_{CAI} is fixed, the CAI value of the designed sequence will stay within a relatively stable range irrespective of any variations in λ_{TIE}.

The regulatory ability of method 100 on mRNA indicators is demonstrated by designing the mRNA sequence of eGFP protein (from GenBank: AFA52650.1). Therefore, five λ_{TIE} parameters (2, 4, 6, 8, 10) and four λ_{CAI} parameters (2, 4, 6, 8) are set, resulting in a total of 20 parameter combinations. The λ_{CAI} parameter accurately adjusts the CAI value of the sequence of interest, as shown in panel A in FIG. 11. As the λ_{CAI} value increases, the CAI value of the designed sequence gradually increases. For each λ_{CAI} value, the CAI value fluctuations of the sequences designed with different λ_{TIE} parameters are very small, indicating that the CAI is mainly regulated by the λ_{CAI} parameters and is almost unaffected by λ_{TIE}, which is consistent with the design expectations of method 100. When λ_{CAI} is fixed, method 100 achieves flexible adjustment of the TIE and MFE indicators of the sequence of interest by changing λ_{TIE}, as shown in panels B and C in FIG. 11. As the λ_{TIE} parameters increase, the TIE value gradually rises, indicating an increase in the translation initiation efficiency of mRNA, while the MFE value also increases, implying a decrease in the structural compactness and thermodynamic stability of the mRNA. As shown in panel D in FIG. 11, there is a positive correlation between TIE and MFE values, indicating a negative correlation between translation initiation efficiency and the structural compactness and thermodynamic stability of the mRNA. In short, the optimization goals can be flexibly customized by adjusting these two hyperparameters to achieve different balances among these three indicators to meet diverse needs in different scenarios.

The present disclosure further provides an mRNA molecule, the sequence of which is prepared by the method, apparatus, electronic device, or computer program product disclosed herein.

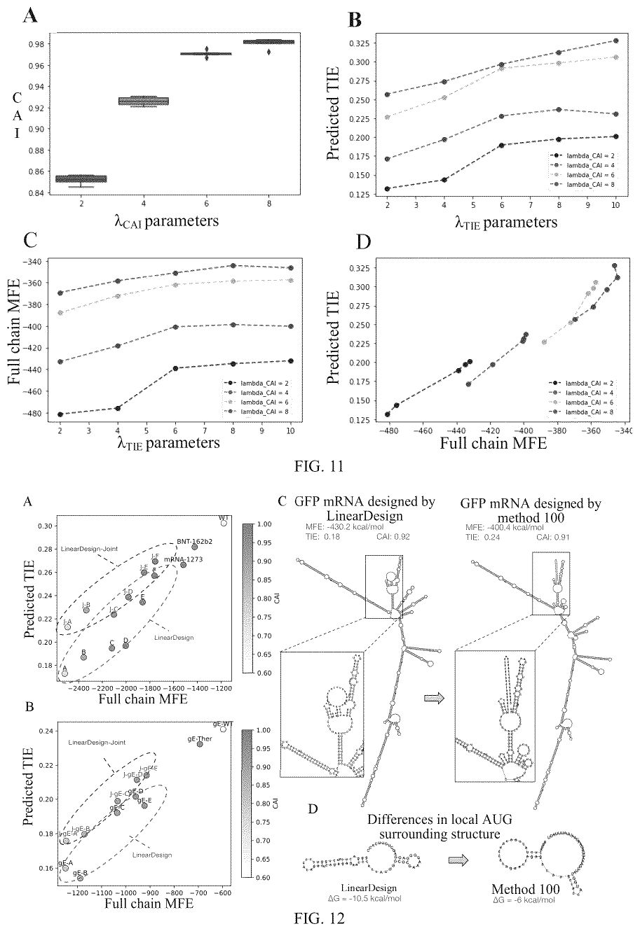

In an embodiment, the LinearDesign algorithm and method 100 are evaluated for the design of the novel coronavirus (SARS-COV-2) spike protein and the varicella-zoster virus (VZV) antigen (VZV gE protein, UniProtKB/Swiss-Prot: Q9J3M8.1), where the amino acid sequences of the novel coronavirus (SARS-COV-2) spike protein and the varicella-zoster virus (VZV) antigen are available from NCBI (National Center for Biotechnology Information). The LinearDesign algorithm is an existing mRNA sequence design algorithm. For the SARS-COV-2 spike protein, as shown in panel A in FIG. 12, the MFE value of the mRNA designed by LinearDesign is significantly lower than that of the wild-type (WT) and commercial vaccine sequences (BNT-162b2 and mRNA-1273) (commercial vaccine sequences are obtained from: https://github.com/NAalytics/Assemblies-of-putative-SARS-COV2-spike-encoding-mRNA-sequences-for-vaccines-BNT-162b2-and-mRNA-1273/), demonstrating significant improvements in structural compactness and thermodynamic stability. However, the TIE values of the sequences designed by LinearDesign are notably lower, indicating a possible reduction in translation efficiency. In contrast, the sequence designed by method 100 has a significantly improved TIE indicator compared with the sequence designed by LinearDesign, while maintaining similar CAI and MFE values. As shown in panel B in FIG. 12, a similar pattern is observed in the design of VZV antigens. Compared with the wild type (gE-WT) and the sequence designed by Thermo Fisher's codon optimization tool (gE-Ther), the sequence designed by LinearDesign has significant advantages in MFE indicator. However, they have obvious shortcomings in TIE indicator. Method 100 effectively addresses this issue by maintaining LinearDesign's advantages in MFE while significantly improving the performance of TIE indicator. This demonstrates that method 100 is capable of producing sequences with superior overall performance in terms of translation efficiency and stability. Given previous analyses of the relationship between metric space and protein expression levels, this improvement is expected to lead to increased protein yields.

There are also significant differences in secondary structure between the sequences designed by method 100 and LinearDesign. Panel C in FIG. 12 shows the secondary structures of the two eGFP mRNA sequences designed by method 100 and LinearDesign, where the original eGFP amino acid sequence is derived from NCBI. Although both sequences show similar MFE and CAI indicators, the sequence designed by method 100 has a significantly better TIE indicator. The main structural differences in the start codon region between the two sequences can be observed. In this region, the sequence designed by Method 100 has fewer hairpin structures and base pairing, resulting in a structurally more relaxed configuration. This looser structure is generally thought to facilitate ribosome binding and scanning in 5′ UTR region, thereby improving translation initiation efficiency. When the sequence of the surrounding region of the start codon is extracted and folded separately, it is also evident that the sequence designed by method 100 has less secondary structure and higher folding free energy, as shown in panel D in FIG. 12. This further supports the technical effect that structural relaxation in the surrounding region of the start codon helps method 100 achieve improved translation initiation efficiency.

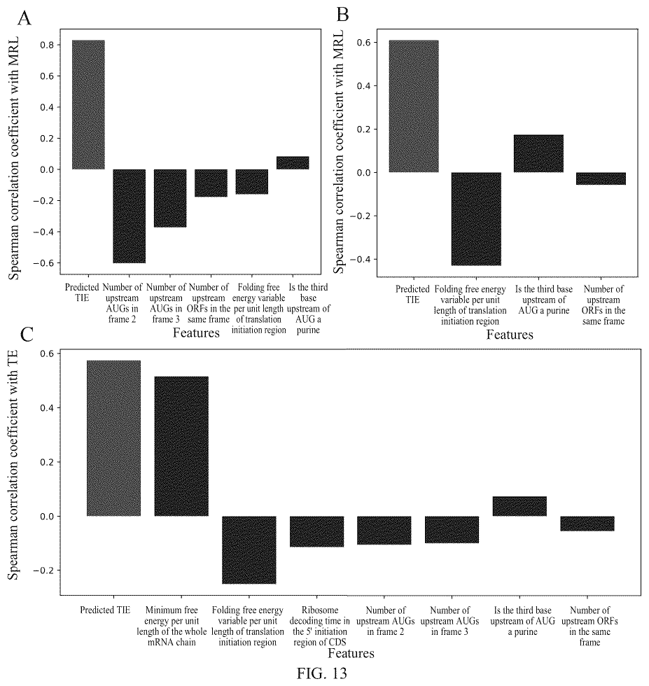

In an embodiment, the accuracy of predicted TIE indicator for eGFP proteins is analyzed using massive parallel translation assay (MPTA) data. The samples in this dataset have fixed CDS and randomly generated 5′ UTR sequences, and the ribosome load of each sequence is measured by multimer analysis. Given that translation elongation efficiency is relatively constant, ribosome loading values reflect the translation initiation efficiency of each sequence. As shown in panel A in FIG. 13, among the top 20,000 samples sorted by read count, the Spearman correlation coefficient between TIE and ribosome load is 0.83, which is significantly higher than other subdivision features. Among the subdivision features, the one associated with the upstream AUG codon (uAUG) has the highest absolute Spearman correlation coefficient with ribosome load. The presence of uAUG may lead to premature translation initiation and inhibit translation of the primary ORF. In actual mRNA vaccine development scenarios, uAUG-containing sequences are generally not used. Therefore, in order to get closer to the real situation, the correlation between various indicators and ribosome load in samples without uAUG is further analyzed. As shown in panel B in FIG. 13, in these samples, the Spearman correlation between TIE and ribosome load reaches 0.6, exceeding the correlation of other subdivision indicators. This demonstrates the robustness of TIE as an indicator of translation initiation efficiency, particularly in the context of a uAUG sequence-free environment.

Since the eGFP protein dataset has a fixed CDS, it cannot effectively reflect the impact of the CDS region on the translation initiation efficiency of mRNA. To address this issue, we performed further analyses using ribosome analysis data from the human PC3 cell line (GSE35469). This dataset contains translation efficiency information for transcripts across the entire human genome. There are significant differences in UTR and CDS sequences between transcripts, making this dataset suitable for analyzing the combined effects of 5′ UTR and CDS on translation initiation efficiency. According to the article “Nicholas T Ingolia, Liana F Lareau, and Jonathan S Weissman. Ribosome profiling of mouse embryonic stem cells reveals the complexity and dynamics of mammalian proteomes. Cell, 147(4): 789-802, 2011”, there are no significant differences in translation elongation efficiency between different genes, and translation initiation efficiency is the major rate-limiting step in the translation process. Therefore, in this context, translation efficiency can be primarily considered as a proxy for translation initiation efficiency.

As shown in panel C in FIG. 13, the Spearman correlation coefficient between translation efficiency and various indicators is presented. The correlation between the predicted TIE indicator and the measured translation efficiency (TE) is 0.574, outperforming other subdivision features. Among other features, the minimum free energy per unit length of the whole mRNA chain (whole_MFE_mean) has the highest correlation with TE, reflecting the significant impact of the structural compactness of the mRNA on translation initiation efficiency. A structure that is too compact may hinder translation initiation. The ribosome residence time in the CDS leader region (i.e., the ribosome decoding time in 5′ initiation region of CDS, CDS_leader_DT) is significantly negatively correlated with TE, because ribosomes staying in the CDS leader region can spatially hinder the assembly of subsequent ribosomes. These correlation patterns further demonstrate that translation initiation efficiency is jointly determined by 5′ UTR and CDS regions.

The present disclosure provides a pharmaceutical composition comprising an mRNA sequence prepared by the method, apparatus, electronic device, or computer program product disclosed herein, or an mRNA molecule disclosed herein, and a pharmaceutically acceptable adjuvant.

The pharmaceutical composition of the present disclosure may be formulated by any means known in the art, including but not limited to formulation as tablets, capsules, caplets, suspensions, powders, lyophilized formulations, suppositories, eye drops, skin patches, oral soluble formulations, sprays, aerosols, and other solid, semi-solid, or liquid system dosage forms.

The pharmaceutical composition may be an immediate release and/or modified release formulation, including delayed release, sustained release, pulse release, controlled release, targeted release, and programmed release formulations.

As used herein, “pharmaceutically acceptable adjuvant” refers to an ingredient in a pharmaceutical composition other than active ingredients that is non-toxic to the subject. Pharmaceutically acceptable adjuvants include, but are not limited to, excipients (such as diluents, carriers, etc.) and additives (such as stabilizers, preservatives, solubilizers, buffers, etc.). Excipients may comprise polyvinylpyrrolidone, gelatin, hydroxypropyl cellulose (HPC), gum arabic, polyethylene glycol, mannitol, sodium chloride, and sodium citrate. For injection formulations or other liquid administration formulations, water containing at least one or more buffer components is preferred, and stabilizers, preservatives, and solubilizers may also be used. For solid administration formulations, any of a variety of thickeners, fillers, extenders, and carrier additives may be employed, such as starches, sugars, cellulose derivatives, fatty acids, and the like. For topical administration formulations, any of a variety of creams, ointments, gels, lotions, and the like may be employed. For most pharmaceutical formulations, the inactive ingredients may constitute a larger portion of the formulation, by weight or volume. For pharmaceutical formulations, it is also contemplated that any of a variety of metered release, slow release, or sustained release formulations and additives may be employed such that the dosage may be formulated to deliver the compounds of the present disclosure over a period of time.

Compounds of the present disclosure may be administered via mucosal, intrabuccal, oral, transdermal, inhaled, intranasal, urethral, and vaginal administration, and intravenous, subcutaneous, intramuscular, and intraperitoneal injection, and other methods of administration. The adjuvants in the pharmaceutical composition are compatible with the route of administration.

In some embodiments, the compound of the present disclosure can be delivered orally, such as in tablets or capsules. The compound may be packaged in an enteric protectant, preferably such that the compound is not released prior to delivery of the tablet or capsule to the stomach, and optionally further to a portion of the small intestine.

In some embodiments, the compounds of the present disclosure may be administered by injection, and pharmaceutical forms suitable for injectable use comprise sterile aqueous solutions or dispersions and sterile powders for the immediate preparation of sterile injectable solutions or dispersions. In all cases, the form must be sterile and must be fluid enough to allow administration by syringe. The form must be stable under the conditions of formulation and storage, and must be preserved against the contaminating action of microorganisms such as bacteria and fungi. The carrier may be a solvent or dispersion medium containing, for example, water, ethanol, polyols (e.g., glycerol, propylene glycol, or liquid polyethylene glycol), suitable mixtures thereof, and vegetable oils.

Therapeutic administration can also be achieved by injection of sustained release formulations, such as those allowing subcutaneous injection, including: nanospheres/microspheres, liposomes, emulsions, gels, insoluble salts, or suspensions.

In some embodiments, the compound of the present disclosure can be administered intranasally. Pharmaceutical compositions may be in the form of aqueous solutions, for example, solutions containing saline, citrate, or other commonly used excipients or preservatives. They may also be available in dry formulation or powder form.

The present disclosure provides the use of an mRNA sequence prepared by the method, apparatus, electronic device, or computer program product disclosed herein, the mRNA molecule disclosed herein, or the pharmaceutical composition disclosed herein in the preparation of a drug or a vaccine.

This method significantly improves the yield and quality of proteins and has important application value in the preparation of a drug or a vaccine.

In some embodiments, the medicine disclosed herein includes, but not limited to: an mRNA drug, a protein replacement therapy medicine, a gene editing medicine, a cancer treatment medicine, a regenerative medicine drug, a DNA gene therapy agent based on a viral or non-viral vector, a modification agent for genetically engineering in an organism, a cell therapy medicine, an enzyme replacement therapy medicine, an aptamer medicine, an microRNA therapy medicine, and ribozyme medicines. In some preferred embodiments, the medicine disclosed herein is selected from an mRNA drug, a DNA gene therapy agent based on a viral or non-viral vector, or a modification agent for genetically engineering in an organism.

In some embodiments, the vaccine disclosed herein are selected from: a preventive mRNA vaccine or a therapeutic mRNA vaccine.

The present disclosure provides a method for treating or preventing a disease, comprising administering to a subject in need thereof an effective amount of an mRNA sequence prepared by the method, apparatus, electronic device, or computer program product disclosed herein, the mRNA molecule disclosed herein, or the pharmaceutical composition disclosed herein.

In some embodiments, the disease disclosed herein includes, but are not limited to, an infectious disease, including a viral infection, such as novel coronavirus and varicella-zoster virus.

As used herein, a “subject” comprises an animal, such as a vertebrate, preferably a mammal, such as a dog, cat, pig, cow, sheep, horse, rodent (e.g., mouse, rat, or guinea pig) or a primate (such as gorilla, chimpanzee, and human).

As used herein, “treat” means alleviating or ameliorating a disease or disorder (i.e., slowing or arresting the progression of a disease or at least one clinical symptom); or alleviating or ameliorating at least one physiological parameter or biomarker associated with the disease or disorder.

As used herein, an “effective amount” is an amount that is sufficient to elicit the desired therapeutic, preventive, or inhibitory effect when administered by any of the above-described means or any other means known in the art and that results in benefiting from it or achieving a certain effect as compared with a corresponding subject who does not receive such amount. This amount is low enough within the scope of sound medical judgment to avoid serious side effects. The effective amount will vary depending on the drug selected, such as mRNA, a pharmaceutical composition, a vaccine; the route of administration; the severity of the disease being treated; and the age, somatotype, weight, and physical condition of a patient to be treated.

According to an embodiment of the present disclosure, there is further provided an electronic device, including: at least one processor; a memory communicatively connected to the at least one processor, where the memory stores instructions that can be executed by the at least one processor, and the instructions, when executed by the at least one processor, cause the at least one processor to perform the method for optimizing an mRNA sequence according to the embodiment of the present disclosure.

According to an embodiment of the present disclosure, there is further provided a non-transient computer-readable storage medium storing computer instructions. The computer instructions are used to cause a computer to perform the method for optimizing an mRNA sequence according to the embodiment of the present disclosure.

According to an embodiment of the present disclosure, there is further provided a computer program product, including computer program instructions, where the computer program instructions, when executed by a processor, cause the method for optimizing an mRNA sequence according to the embodiment of the present disclosure to be implemented.

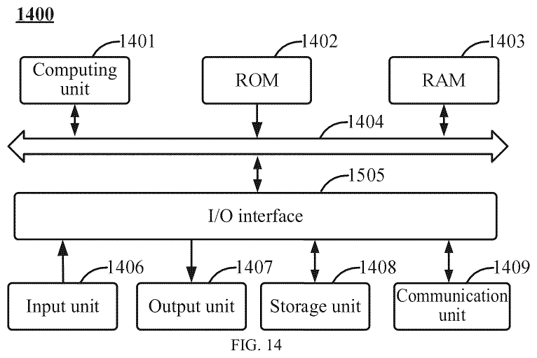

Referring to FIG. 14, a block diagram of a structure of an electronic device 1400 that can serve as a server or a client of the present disclosure is now described. The electronic device is an example of a hardware device that can be applied to various aspects of the present disclosure. The electronic device is intended to represent various forms of digital electronic computer devices, such as a laptop computer, a desktop computer, a workstation, a personal digital assistant, a server, a blade server, a mainframe computer, and other suitable computers. The electronic device may further represent various forms of mobile apparatuses, such as a personal digital assistant, a cellular phone, a smartphone, a wearable device, and other similar computing apparatuses. The components shown in the present specification, their connections and relationships, and their functions are merely examples, and are not intended to limit the implementation of the present disclosure described and/or required herein.

As shown in FIG. 14, the electronic device 1400 includes a computing unit 1401. The computing unit may perform various appropriate actions and processing according to a computer program stored in a read-only memory (ROM) 1402 or a computer program loaded from a storage unit 1408 to a random access memory (RAM) 1403. The RAM 1403 may further store various programs and data required for the operation of the electronic device 1400. The computing unit 1401, the ROM 1402, and the RAM 1403 are connected to each other through a bus 1404. An input/output (I/O) interface 1405 is also connected to the bus 1404.

A plurality of components in the electronic device 1400 are connected to the I/O interface 1405, including: an input unit 1406, an output unit 1407, the storage unit 1408, and a communication unit 1409. The input unit 1406 may be any type of device capable of entering information to the electronic device 1400. The input unit 1406 may receive entered digit or character information, and generate a key signal input related to user settings and/or function control of the electronic device, and may include, but is not limited to, a mouse, a keyboard, a touchscreen, a trackpad, a trackball, a joystick, a microphone, and/or a remote controller. The output unit 1407 may be any type of device capable of presenting information, and may include, but is not limited to, a display, a speaker, a video/audio output terminal, a vibrator, and/or a printer. The storage unit 1408 may include, but is not limited to, a magnetic disk and an optical disk. The communication unit 1409 allows the electronic device 1400 to exchange information/data with other devices through a computer network such as the Internet and/or various telecommunication networks, and may include, but is not limited to, a modem, a network interface card, an infrared communication device, a wireless communication transceiver, and/or a chipset, for example, a Bluetooth device, an 802.11 device, a Wi-Fi device, a WiMAX device, a cellular communication device and/or the like.

The computing unit 1401 may be various general-purpose and/or special-purpose processing components with processing and computing capabilities. Some examples of the computing unit 1401 include, but are not limited to, a central processing unit (CPU), a graphics processing unit (GPU), various dedicated artificial intelligence (AI) computing chips, various computing units that run machine learning model algorithms, a digital signal processor (DSP), and any appropriate processor, controller, microcontroller, etc. The computing unit 1401 performs the various methods and processing described above, for example, the method 100. For example, in some embodiments, the method 100 may be implemented as a computer software program, which is tangibly contained in a machine-readable medium, such as the storage unit 1408. In some embodiments, a part or all of the computer program may be loaded and/or installed onto the electronic device 1400 via the ROM 1402 and/or the communication unit 1409. When the computer program is loaded into the RAM 1403 and executed by the computing unit 501, one or more steps of the method 100 described above can be performed. Alternatively, in other embodiments, the computing unit 1401 may be configured, by any other appropriate means (for example, by means of firmware), to perform the method 100.

Various implementations of the systems and technologies described herein above can be implemented in a digital electronic circuit system, an integrated circuit system, a field programmable gate array (FPGA), an application-specific integrated circuit (ASIC), an application-specific standard product (ASSP), a system-on-chip (SOC) system, a complex programmable logical device (CPLD), computer hardware, firmware, software, and/or a combination thereof. These various implementations may include: implementation in one or more computer programs, where the one or more computer programs may be executed and/or interpreted on a programmable system including at least one programmable processor. The programmable processor may be a dedicated or general-purpose programmable processor that can receive data and instructions from a storage system, at least one input apparatus, and at least one output apparatus, and transmit data and instructions to the storage system, the at least one input apparatus, and the at least one output apparatus.

Program codes used to implement the method of the present disclosure can be written in any combination of one or more programming languages. The program code may be provided for a processor or a controller of a general-purpose computer, a special-purpose computer, or other programmable data processing apparatuses, such that when the program code is executed by the processor or the controller, the functions/operations specified in the flowcharts and/or block diagrams are implemented. The program code may be completely executed on a machine, or partially executed on a machine, or may be, as an independent software package, partially executed on a machine and partially executed on a remote machine, or completely executed on a remote machine or a server.

In the context of the present disclosure, the machine-readable medium may be a tangible medium, which may contain or store a program for use by an instruction execution system, apparatus, or device, or for use in combination with the instruction execution system, apparatus, or device. The machine-readable medium may be a machine-readable signal medium or a machine-readable storage medium. The machine-readable medium may include, but is not limited to, an electronic, magnetic, optical, electromagnetic, infrared, or semiconductor system, apparatus, or device, or any suitable combination thereof. More specific examples of the machine-readable storage medium may include an electrical connection based on one or more wires, a portable computer disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof.

In order to provide interaction with a user, the systems and technologies described herein can be implemented on a computer which has: a display apparatus (for example, a cathode-ray tube (CRT) or a liquid crystal display (LCD) monitor) configured to display information to the user; and a keyboard and a pointing apparatus (for example, a mouse or a trackball) through which the user can provide an input to the computer. Other categories of apparatuses can also be used to provide interaction with the user; for example, feedback provided to the user can be any form of sensory feedback (for example, visual feedback, auditory feedback, or tactile feedback); and an input from the user can be received in any form (including an acoustic input, a voice input, or a tactile input).

The systems and technologies described herein can be implemented in a computing system (for example, as a data server) including a backend component, or a computing system (for example, an application server) including a middleware component, or a computing system (for example, a user computer with a graphical user interface or a web browser through which the user can interact with the implementation of the systems and technologies described herein) including a frontend component, or a computing system including any combination of the backend component, the middleware component, or the frontend component. The components of the system can be connected to each other through digital data communication (for example, a communication network) in any form or medium. Examples of the communication network include: a local area network (LAN), a wide area network (WAN), the Internet, and a blockchain network.

A computer system may include a client and a server. The client and the server are generally far away from each other and usually interact through a communication network. A relationship between the client and the server is generated by computer programs running on respective computers and having a client-server relationship with each other. The server may be a cloud server, a server in a distributed system, or a server combined with a blockchain.

It should be understood that steps may be reordered, added, or deleted based on the various forms of procedures shown above. For example, the steps recorded in the present disclosure may be performed in parallel, in order, or in a different order, provided that the desired result of the technical solutions disclosed in the present disclosure can be achieved, which is not limited herein.

Although the embodiments or examples of the present disclosure have been described with reference to the drawings, it should be understood that the methods, systems and devices described above are merely exemplary embodiments or examples, and the scope of the present disclosure is not limited by the embodiments or examples, and is only defined by the scope of the granted claims and the equivalents thereof. Various elements in the embodiments or examples may be omitted or substituted by equivalent elements thereof. Moreover, the steps may be performed in an order different from that described in the present disclosure. Further, various elements in the embodiments or examples may be combined in various ways. It is important that, as the technology evolves, many elements described herein may be replaced with equivalent elements that appear after the present disclosure.

What is claimed is:

1. A method for optimizing a messenger ribonucleotide (mRNA) sequence, comprising:
obtaining a first mRNA sequence for synthesizing a protein of interest, wherein the first mRNA sequence comprises a 5′ untranslated region sequence and a coding region sequence; and
adjusting 5′ untranslated region sequence and the coding region sequence with the goal of maximizing a first score of the first mRNA sequence, so as to obtain an optimized second mRNA sequence for synthesizing the protein of interest, wherein the first score reflects at least one of the following indicators of the first mRNA sequence: translation initiation efficiency, codon adaptation index, and minimum free energy.

2. The method according to claim 1, wherein the obtaining the first mRNA sequence for synthesizing the protein of interest comprises:
obtaining a preset untranslated region sequence library, wherein the untranslated region sequence library comprises at least one candidate 5′ untranslated region sequence, and each candidate 5′ untranslated region sequence in the at least one candidate 5′ untranslated region sequence enables gene expression; and
the 5′ untranslated region sequence is determined from the at least one candidate 5′ untranslated region sequence.

3. The method according to claim 1, wherein the obtaining the first mRNA sequence for synthesizing the protein of interest comprises:
generating an initial coding region sequence corresponding to the amino acid sequence of the protein of interest; and
adjusting the initial coding region sequence with the goal of maximizing a second score of the initial coding region sequence, so as to obtain the coding region sequence, wherein the second score reflects the codon adaptation index and/or minimum free energy of the initial coding region sequence.

4. The method according to claim 1, wherein the adjusting 5′ untranslated region sequence and the coding region sequence with the goal of maximizing a first score of the first mRNA sequence, so as to obtain an optimized second mRNA sequence for synthesizing the protein of interest comprises:
obtaining at least one third mRNA sequence by mutating 5′ untranslated region sequence and the coding region sequence;
calculating a first score for each of the at least one third mRNA sequence; and
determining the third mRNA sequence with the highest first score as the second mRNA sequence.

5. The method according to claim 1, wherein the adjusting 5′ untranslated region sequence and the coding region sequence with the goal of maximizing a first score of the first mRNA sequence, so as to obtain an optimized second mRNA sequence for synthesizing the protein of interest comprises:
splitting 5′ untranslated region sequence and the coding region sequence into a translation initiation region sequence and a coding region main sequence, wherein the translation initiation region sequence comprises at least 5′ untranslated region sequence, and the coding region main sequence comprises nucleotides in the coding region sequence that are not included in the translation initiation region sequence;
adjusting the translation initiation region sequence with the goal of maximizing the translation initiation efficiency of the first mRNA sequence, so as to obtain a fourth mRNA sequence; and
adjusting the coding region main sequence of the fourth mRNA sequence with the goal of maximizing a first score of the fourth mRNA sequence, so as to obtain the second mRNA sequence.

6. The method according to claim 5, wherein the translation initiation region sequence comprises 5′ untranslated region sequence and a preset number of nucleotides in the coding region sequence close to 5′ untranslated region sequence.

7. The method according to claim 5, wherein the adjusting the translation initiation region sequence with the goal of maximizing the translation initiation efficiency of the first mRNA sequence, so as to obtain a fourth mRNA sequence comprises:
obtaining at least one fifth mRNA sequence by mutating the translation initiation region sequence;
calculating the translation initiation efficiency of each of the at least one fifth mRNA sequence; and
determining the fifth mRNA sequence with the greatest translation initiation efficiency as the fourth mRNA sequence.

8. The method according to claim 7, wherein the calculating the translation initiation efficiency of each of the at least one fifth mRNA sequence comprises:
for each fifth mRNA sequence of the at least one fifth mRNA sequence:
extracting a feature for predicting the translation initiation efficiency of the fifth mRNA sequence; and
inputting the feature into a trained translation initiation efficiency prediction model to obtain the translation initiation efficiency of the fifth mRNA sequence output by the translation initiation efficiency prediction model.

9. The method according to claim 8, wherein the feature comprises at least one of the following:
a structural compactness of the translation initiation region sequence, a whole structural compactness, a Kozak sequence feature, an upstream start codon and upstream open reading frame sequence feature, and a ribosome residence time of a leading region of the coding region sequence.

10. The method according to claim 5, wherein the adjusting the coding region main sequence of the fourth mRNA sequence with the goal of maximizing a first score of the fourth mRNA sequence, so as to obtain the second mRNA sequence comprises:
obtaining at least one sixth mRNA sequence by mutating the coding region main sequence of the fourth mRNA sequence;
calculating a first score for each of the at least one sixth mRNA sequence; and
determining the sixth mRNA sequence with the highest first score as the second mRNA sequence.

11. The method according to claim 10, wherein the determining the sixth mRNA sequence with the highest first score as the second mRNA sequence comprises:
determining the sixth mRNA sequence as the second mRNA sequence in response to the codon adaptation index of the sixth mRNA sequence with the highest first score being greater than a threshold, wherein the threshold is determined based on the codon adaptation index of the first mRNA sequence.

12. An mRNA molecule, wherein the sequence of the mRNA molecule is prepared by a method for optimizing an mRNA sequence, wherein the method for optimizing an mRNA sequence comprises:
obtaining a first mRNA sequence for synthesizing a protein of interest, the first mRNA sequence comprises a 5′ untranslated region sequence and a coding region sequence;
and adjusting 5′ untranslated region sequence and the coding region sequence with the goal of maximizing a first score of the first mRNA sequence, so as to obtain an optimized second mRNA sequence for synthesizing the protein of interest, the first score reflects at least one of the following indicators of the first mRNA sequence: translation initiation efficiency, codon adaptation index, and minimum free energy.

13. A pharmaceutical composition, comprising the mRNA molecule according to claim 12 and a pharmaceutically acceptable adjuvant.

14. A method of treatment or prevention of diseases, comprising administering an effective amount of the mRNA molecule according to claim 13.

15. The method according to claim 14, wherein the diseases are selected from infectious diseases or cancers.

16. A non-transient computer-readable storage medium storing computer instructions, wherein the computer instructions are used to cause a computer to perform operations comprising:
obtaining a first mRNA sequence for synthesizing a protein of interest, wherein the first mRNA sequence comprises a 5′ untranslated region sequence and a coding region sequence; and
adjusting 5′ untranslated region sequence and the coding region sequence with the goal of maximizing a first score of the first mRNA sequence, so as to obtain an optimized second mRNA sequence for synthesizing the protein of interest, wherein the first score reflects at least one of the following indicators of the first mRNA sequence: translation initiation efficiency, codon adaptation index, and minimum free energy.
